SEQUENCE LISTING (12 sequences)

SEQ ID NO: 1 - protein, 107 aa, Homo sapiens [sequence omitted]

SEQ ID NO: 2 - protein, 214 aa, Homo sapiens
DIQMTQSPSSLSASVGDRVTITCGTSEDIINYLNWYQQKPGKAPKLLIYHTSRLQSGVPSRFSGSGSGTDFTLTISSLQPEDFATYYCQQGYTLPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 121 aa, Homo sapiens [sequence omitted]

SEQ ID NO: 4 - protein, 451 aa, Homo sapiens
EVQLVQSGAEVKKPGASVKVSCKASGYTFTSYVIHWVRQRPGQGLAWMGYINPYNDGTKYNERFKGKVTITSDRSTSTVYMELSSLRSEDTAVYLCGREGIRYYGLLGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 400 aa, Homo sapiens
DLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWFVIVIMATICFILLILSLICKICHLWIKLFPPIPAPKSNIKDLFVTTNYEKAGSSETEIEVICYIEKPGVETLEDSVF

SEQ ID NO: 6 - protein, 398 aa, Mus musculus
DLLNHKKFLLLPPVNFTIKATGLAQVLLHWDPNPDQEQRHVDLEYHVKINAPQEDEYDTRKTESKCVTPLHEGFAASVRTILKSSHTTLASSWVSAELKAPPGSPGTSVTNLTCTTHTVVSSHTHLRPYQVSLRCTWLVGKDAPEDTQYFLYYRFGVLTEKCQEYSRDALNRNTACWFPRTFINSKGFEQLAVHINGSSKRAAIKPFDQLFSPLAIDQVNPPRNVTVEIESNSLYIQWEKPLSAFPDHCFNYELKIYNTKNGHIQKEKLIANKFISKIDDVSTYSIQVRAAVSSPCRMPGRWGEWSQPIYVGKERKSLVEWHLIVLPTAACFVLLIFSLICRVCHLWTRLFPPVPAPKSNIKDLPVVTEYEKPSNETKIEVVHCVEEVGFEVMGNSTF

SEQ ID NO: 7 - protein, 5 aa, Homo sapiens
SYVIH

SEQ ID NO: 8 - protein, 17 aa, Homo sapiens
YINPYNDGTKYNERFKG

SEQ ID NO: 9 - protein, 12 aa, Homo sapiens
EGIRYYGLLGDY

SEQ ID NO: 10 - protein, 11 aa, Homo sapiens
GTSEDIINYLN

SEQ ID NO: 11 - protein, 7 aa, Homo sapiens
HTSRLQS

SEQ ID NO: 12 - protein, 9 aa, Homo sapiens
QQGYTLPYT

(19) United States
(12) Patent Application Publication
(10) Pub. No.: US 2021/0095037 A1
(43) Pub. Date: Apr. 1, 2021

(54) METHODS FOR TREATING LATE-ONSET ASTHMA USING BENRALIZUMAB

(71) Applicant: ASTRAZENECA AB, Södertälje (SE)

(72) Inventors: Ian HIRSCH, Gaithersburg, MD (US); Paul NEWBOLD, Gaithersburg, MD (US)

(21) Appl. No.: 17/031,965

(22) Filed: Sep. 25, 2020

Related U.S. Application Data
(60) Provisional application No. 62/907,270, filed on Sep. 27, 2019.

(51) Int. Cl. C07K 16/28; A61P 11/06; A61K 9/00; A61K 45/06; A61K 39/395

(52) U.S. Cl. CPC C07K 16/2866; A61P 11/06; A61K 9/0019; A61K 2039/545; A61K 45/06; A61K 39/3955; A61K 9/0029

(57) ABSTRACT

Provided herein are methods of treating patients with late-onset asthma or asthma falling within Severe Asthma Research Program (SARP) clinical cluster 3 or 5 comprising administering to the patient a therapeutically effective amount of the anti-interleukin-5 receptor (IL-5R) antibody, benralizumab, or an antigen-binding fragment thereof. Also provided are methods of predicting an enhanced therapeutic response to benralizumab by determining the SARP clinical cluster of the patient's asthma prior to administration.

### SEQUENCE LISTING

The instant application contains a Sequence Listing which has been submitted electronically in ASCII format and is hereby incorporated by reference in its entirety. Said ASCII copy, created on Aug. 31, 2020, is named IL5R-611-US-NP_SL.txt and is 15,946 bytes in size.

### BACKGROUND

Asthma is a heterogeneous respiratory disease affecting 1-18% of the population in different countries. It is typically characterized by chronic airway inflammation and a history of respiratory symptoms such as wheeze, shortness of breath, chest tightness, and cough that vary over time and in intensity, together with variable expiratory airflow limitation. Asthma may be categorized into different phenotypes based on distinct, recognizable clusters of demographic, clinical, and/or pathophysiological characteristics; however, these do not correlate strongly with specific pathological processes or treatment responses. See, Global Initiative for Asthma. Global Strategy for Asthma Management and Prevention (2019), available from www.ginasthma.org, accessed September 2019.

In 2010, the National Heart, Lung, and Blood Institute Severe Asthma Research Program (SARP) identified five distinct clinical phenotypes of asthma (i.e., Cluster 1, 2, 3, 4, and 5) using an unsupervised hierarchical cluster analysis of 34 phenotypic variables in 726 subjects with asthma. See, Moore, W. C. et al., Am J Respir Crit Care Med. 181:315-323 (2010). The variables covered a broad spectrum of routine assessments of asthma patients, such as, demographic data (e.g., sex, race, age); physiologic measures (e.g., lung function and atopy); and additional variables effecting disease severity (e.g., age of onset and duration). Moore also conducted a discriminant analysis using the 34 variables and identified 11 of the strongest discriminatory variables for cluster assignment. Additionally, Moore reported on an even simpler algorithm using only 3 variables for asthma cluster assignment with about 80% accuracy. The specific phenotypic variables are described further below.

Benralizumab is a humanized monoclonal antibody (mAb) that binds to the alpha chain of the interleukin-5 receptor alpha (IL-5Rα), which is expressed on eosinophils and basophils. It induces apoptosis of these cells via antibody-dependent cell cytotoxicity. Two Phase III clinical trials, SIROCCO (ClinicalTrials.gov identifier NCT01928771) and CALIMA (ClinicalTrials.gov identifier NCT01914757), demonstrated that benralizumab, in combination with high-dosage inhaled corticosteroids/long-acting β2-agonists (ICS/LABA), significantly reduced asthma exacerbations and improved lung function and disease control for patients with severe, uncontrolled asthma and blood eosinophil counts ≥300 cells/μL versus placebo. See, Bleeker, E. R. et al., Lancet 388:2115-2127 (2016)) and FitzGerald, J. M. et al., Lancet 388:2128-2141 (2016), respectively. Benralizumab 30 mg subcutaneous formulation administered every 8 weeks (Q8W, first three doses every 4 weeks (Q4W)) was subsequently approved in several markets as add-on maintenance treatment for patients with severe, uncontrolled eosinophilic asthma. See, AstraZeneca. Fasenra™ (Benralizumab), Prescribing Information (2017), www.azpicentral.com/fasenra/fasenra_pi.pdf, date last updated: November, 2017; date last accessed: Jan. 10, 2018; AstraZeneca. Fasenra™ (Benralizumab), Summary of Product Characteristics (2018), http://ec.europa.eu/health/documents/community-register/2018/20180108139598/anx_139598_en.pdf; date last accessed: Mar. 13, 2018.

Understanding asthma heterogeneity is important for determining responsiveness to targeted therapies and the development of phenotype-guided therapies for treating patients with severe asthma. While benralizumab significantly reduces asthma exacerbations and improves lung function in severe uncontrolled asthma, there is an unmet need for determining if asthma clinical phenotypes impact the clinical efficacy of benralizumab, and to identify those phenotypes in order to inform clinical decisions.

### BRIEF SUMMARY

As described herein, it has been discovered, through analyses of over 2,200 pooled patients from the SIROCCO and CALIMA trials, that identifying certain subsets or subpopulations of asthma patients using benralizumab has the potential for predicting an enhanced response to treatment with benralizumab.

In certain aspects, methods of using benralizumab or an antigen-binding fragment thereof are as provided in FIGS. 1-5 and Example 1.

In certain aspects, a method of treating a patient with late-onset asthma comprises administering to the patient a therapeutically effective amount of benralizumab or an antigen-binding fragment thereof

In certain aspects, a method of treating a patient with asthma that falls within Severe Asthma Research Program (SARP) clinical cluster 3 or 5 comprises administering to the patient a therapeutically effective amount of benralizumab or an antigen-binding fragment thereof.

In certain aspects, a method of reducing the annual exacerbation rate (AER) in a patient with late-onset asthma comprises administering to the patient a therapeutically effective amount of benralizumab or an antigen-binding fragment thereof, wherein the administration reduces the patient's AER.

In certain aspects, a method of reducing the AER in a patient with asthma that falls within SARP clinical cluster 3 comprises administering to the patient a therapeutically effective amount of benralizumab or an antigen-binding fragment thereof, wherein the administration reduces the patient's AER.

In certain aspects, a method of reducing the AER in a patient with asthma that falls within SARP clinical cluster 5 comprises administering to the patient a therapeutically effective amount of benralizumab or an antigen-binding fragment thereof, wherein the administration reduces the patient's AER.

In certain aspects of a method provided herein, the AER is reduced by at least 45% compared to a patient not administered the benralizumab or antigen-binding fragment thereof. In certain aspects, the AER is reduced by at least 50% compared to a patient not administered the benralizumab or antigen-binding fragment thereof.

In certain aspects, a method of improving lung function in a patient with late-onset asthma comprises administering to the patient a therapeutically effective amount of benralizumab or an antigen-binding fragment thereof.

In certain aspects, a method of improving lung function in a patient with asthma that falls within SARP clinical cluster 3 comprises administering to the patient a therapeutically effective amount of benralizumab or an antigen-binding fragment thereof.

In certain aspects, a method of improving lung function in a patient with asthma that falls within SARP clinical cluster 5 comprises administering to the patient a therapeutically effective amount of benralizumab or an antigen-binding fragment thereof.

In certain aspects of a method provided herein, the improved lung function is measured by an increase in the patient's percent predicted forced expiratory volume in 1 second (FEV₁) compared to the patient's FEV₁ prior to the administration. In certain aspects, the FEV₁ is pre-bronchodilator (BD) FEV₁. In certain aspects, the pre-BD FEV₁ is increased by at least 6%. In certain aspects, the pre-BD FEV₁ is increased by at least 14%. In certain aspects, the FEV₁ is post-bronchodilator (BD) FEV₁. In certain aspects, the post-BD FEV₁ is increased by at least 2%. In certain aspects, the post-BD FEV₁ is increased by at least 10%.

In certain aspects of a method provided herein, the improved lung function is measured by an increase in the patient's percent predicted forced vital capacity (FVC) compared to the patient's FVC prior to the administration. In certain aspects, the FVC is pre-bronchodilator (BD) FVC. In certain aspects, the pre-BD FVC is increased by at least 6%. In certain aspects, the pre-BD FVC is increased by at least 12%. In certain aspects, the FVC is post-BD FVC. In certain aspects, the post-BD FVC is increased by at least 1%. In certain aspects, the post-BD FVC is increased by at least 7%.

In certain aspects of a method provided herein, the SARP clinical cluster has been determined for the patient's asthma prior to the administration.

In certain aspects of a method provided herein, the method further comprises determining the SARP clinical cluster of the patient's asthma prior to the administration.

In certain aspects of a method provided herein, the determination of the SARP clinical cluster is based on the age of asthma onset, pre-BD FEV₁, and post-BD FEV₁.

In certain aspects of a method provided herein, the patient's age of asthma onset is 47±9.4 years, baseline FEV₁ is 66±7.7%, and maximal post-BD FEV₁ is 78±12%.

In certain aspects of a method provided herein, the patient's age of asthma onset is 33±17.0 years, baseline FEV₁ is 43±9.4%, and maximal post-BD FEV₁ is 56±15%.

In certain aspects of a method provided herein, the determination of the SARP clinical cluster is based on FEV₁, FVC, FEV₁/FVC, maximal post-BD FEV₁, maximal post-BD FVC, percentage change in post-BD FEV₁, age at asthma onset, asthma duration, patient gender, frequency of β2-agonist use, and inhaled corticosteroid (ICS) dosage.

In certain aspects of a method provided herein, the age of asthma onset is 47±9.4 years; the patient's baseline FEV₁ is 66±7.7%; the patient's baseline FVC is 82±11%; the patient's baseline FEV₁/FVC is 0.65±0.10; the patient's maximal post-BD FEV₁ is 78±12%; the patient's maximal post-BD FVC % is 91±14%; the patient's change in post-BD FEV₁ is 0.22+/−0.40; and/or the asthma has had a duration of 10±7 years.

In certain aspects of a method provided herein, the age of asthma onset is 33±17.0; the patient's baseline FEV₁ is 43±9.4; the patient's baseline FVC is 65±12%; the patient's baseline FEV₁/FVC is 0.54±0.12; the patient's maximal post-BD FEV₁ is 56±15%; the patient's maximal post-BD FVC % is 77±15%; the patient's change in post-BD FEV₁ is 0.50+/−0.55; and/or the asthma has had a duration of 21±15 years.

In certain aspects of a method provided herein, the patient has a baseline blood eosinophil count of >300 cells/4 prior to the administration.

In certain aspects of a method provided herein, AER, FEV₁, and FVC values are improved in the patient compared to patients not administered the benralizumab or an antigen-binding fragment thereof.

In certain aspects of a method provided herein, the patient has severe asthma.

In certain aspects of a method provided herein, severe asthma is characterized by a requirement for treatment with high-dose ICSs and/or treatment with continuous or near continuous oral corticosteroids (OCs); and two or more of the following criteria: a requirement for additional daily treatment with a controller medication (e.g., long-acting 132 agonist (LABA), theophylline, or leukotriene antagonist); asthma symptoms requiring short-acting132 agonist (SABA) use on a daily or near-daily basis; persistent airway obstruction (FEV₁<80% predicted, diurnal peak expiratory flow variability >20%); one or more urgent care visits for asthma per year; three or more oral steroid bursts per year; prompt deterioration with a <25% reduction in oral or inhaled corticosteroid dose; and/or near-fatal asthma event in the past.

In certain aspects of a method provided herein, the benralizumab or antigen-binding fragment thereof is administered at about 30 mg per dose.

In certain aspects of a method provided herein, the method comprises at least two doses of the benralizumab or an antigen-binding fragment thereof to the patient.

In certain aspects of a method provided herein, the benralizumab or antigen-binding fragment thereof is administered once every four weeks or once every eight weeks. In certain aspects of a method provided herein, the benralizumab or antigen-binding fragment thereof is administered once every four weeks. In certain aspects of a method provided herein, the benralizumab or antigen-binding fragment thereof is administered once every four weeks for twelve weeks and then once every eight weeks.

In certain aspects of a method provided herein, the benralizumab or antigen-binding fragment thereof is administered parenterally. In certain aspects, the benralizumab or antigen-binding fragment thereof is administered subcutaneously.

In certain aspects of a method provided herein, the benralizumab or antigen-binding fragment thereof is administered in addition to corticosteroid therapy and/or short- or long-acting B2-agonist therapy.

In certain aspects of a method provided herein, the patient has an asthma control questionnaire score of at least 1.5 prior to the administration of benralizumab or antigen-binding fragment thereof.

In certain aspects, a method of predicting an asthma patient's therapeutic response to benralizumab or an antigen-binding fragment thereof comprises determining, prior to administration of the benralizumab or antigen-binding fragment thereof, the SARP clinical cluster of the asthma. In certain aspects, the method comprises predicting an enhanced response to the benralizumab or antigen-binding fragment thereof if the SARP clinical cluster is determined to be cluster 3 or cluster 5. In certain aspects, the method further comprises administering the benralizumab or antigen-binding fragment thereof to the patient if the SARP clinical cluster of the patient's asthma is determined to be cluster 3 or cluster 5.

### BRIEF DESCRIPTION OF THE DRAWINGS/FIGURES

FIG. 1 shows the SARP asthma clinical clusters, i.e., Cluster 1, 2, 3, 4, and 5, along with representative clinical, demographic, and/or pathophysiological characteristics for each cluster.

FIG. 2 is a pie chart showing the SARP cluster distribution, by percentage and total number, of patients from the SIROCCO and CALIMA (N=2,281) phase III clinical trials on benraluzimab. Patients met criteria for Clusters 2, 3, 4, and 5.

FIGS. 3A-3B are bar graphs showing the annual exacerbation rate (AER) for combined benraluzimab arms (using the combined data for benralizumab 30 mg every 4 weeks (Q4W) and 30 mg every 8 weeks (first three doses were Q4W) treatment groups) vs. placebo (all eosinophil counts). FIG. 3A indicates the AER and standard deviation (SD) by cluster vs. placebo. “b” indicates p>0.05; and “c” indicates p<0.0001. FIG. 3B indicates the percentage decrease in AER by cluster vs. placebo after one year of treatment with benralizumab.

FIG. 4 presents bar graphs showing lung function improvement as measured by forced expiratory volume in 1 second (FEV₁) (% predicted normal) before and after treatment by cluster assignment for patients receiving benraluzimab (using the combined data for benralizumab 30 mg every 4 weeks (Q4W) and 30 mg every 8 weeks (first three doses Q4W) treatment groups) (all eosinophil counts). For all comparisons of pre- and post-bronchodilator (BD) values, Kruskal-Wallis p<0.0001 was used, both at baseline and after benralizumab treatment.

FIG. 5 presents bar graphs showing lung function improvement as measured by forced vital capacity (FVC) (% predicted normal) before and after treatment by cluster assignment for patients receiving benraluzimab (using the combined data for benralizumab 30 mg every 4 weeks (Q4W) and 30 mg every 8 weeks (first three doses Q4W) treatment groups) (all eosinophil counts).

### DETAILED DESCRIPTION

### Definitions

The use of the terms “a,” “an,” and “at least one,” refer to one or more of that entity, unless otherwise indicated herein or clearly contradicted by context. For example, “an anti-IL-5α antibody” is understood to represent one or more anti-IL-5α antibodies. As such, the terms “a” (or “an”), “one or more,” and “at least one” can be used interchangeably herein.

The term “about” when used in reference to numerical ranges, cutoffs, or specific values is used to indicate that the recited values may vary by up to as much as 10% from the listed value. As many of the numerical values used herein are experimentally determined, it should be understood by those skilled in the art that such determinations can, and often times will, vary among different experiments. The values used herein should not be considered unduly limiting by virtue of this inherent variation. Thus, the term “about” is used to encompass variations of ±10% or less, variations of ±5% or less, variations of ±1% or less, variations of ±0.5% or less, or variations of ±0.1% or less from the specified value.

The terms “comprising,” “having,” “including,” and “containing” are to be construed as open-ended terms (i.e., meaning “including, but not limited to,”) unless otherwise noted.

The term “and/or” as used in a phrase such as “A and/or B” herein is intended to include the following embodiments: “A and B,” “A or B,” “A,” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Unless specifically stated or obvious from context, as used herein, the term “or” is understood to be inclusive.

As used herein, “treating” and like terms refer to reducing the severity and/or frequency of asthma symptoms, eliminating asthma symptoms and/or the underlying cause of the symptoms, reducing the frequency or likelihood of asthma symptoms and/or their underlying cause, and improving or remediating damage caused, directly or indirectly, by asthma.

As used herein, a “therapeutically effective dose” refers to an amount of benralizumab, as described herein, effective to achieve a particular biological or therapeutic result such as, but not limited to, biological or therapeutic results disclosed or exemplified herein.

As used herein, the terms “patient” and “subject” are used interchangeably and refer to members of the animal kingdom including but not limited to human beings. In a preferred embodiment, the patient is human.

As used herein, the term “late-onset asthma” refers to an asthma diagnosis in a patient that was at least 16 years old at the time of initial diagnosis. In some embodiments, the patient with “late-onset asthma” is from 16 to 56 years old or from 33 to 47 years old at the time of initial diagnosis. For example, in some embodiments, a patient diagnosed with asthma at age 16, 33, 47, or 56 would be considered to have “late-onset asthma.”

As used herein, unless otherwise stated, the term “severe asthma” is asthma characterized by the American Thoracic Society's (ATS) workshop consensus. For example, severe asthma is characterized by a requirement for treatment with high-dose ICSs and/or treatment with continuous or near continuous oral corticosteroids (OCs); and two or more of the following criteria: a requirement for additional daily treatment with a controller medication (e.g., LABA, theophylline, or leukotriene antagonist); asthma symptoms requiring SABA use on a daily or near-daily basis; persistent airway obstruction (FEV₁<80% predicted, diurnal peak expiratory flow variability >20%); one or more urgent care visits for asthma per year; three or more oral steroid bursts per year; prompt deterioration with a ≤25% reduction in oral or inhaled corticosteroid dose; and/or near-fatal asthma event in the past. See, Moore et al., J Allergy Clin Immunol 119:405-413 (2007).

As used herein, “SARP clinical clusters” or “SARP clusters” refer to the five asthma clinical phenotypic clusters (i.e., clusters 1, 2, 3, 4, and 5) identified by the National Heart, Lung, and Blood Institute's Severe Asthma Research Program (SARP) and described in Moore, W. C. et al., Am J Respir Crit Care Med. 181:315-323 (2010), which is incorporated by reference in its entirety. As used herein, asthma patients grouped into SARP clinical cluster 3 and clinical cluster 5 are also said to have “late-onset asthma.”

As used herein, “forced expiratory volume in 1 second (FEV₁)” refers to the maximal amount of air that can forcefully be exhaled in one second. As described herein, FEV₁ can be measured at baseline, e.g., baseline values determined pre- or post-bronchodilator (BD) administration, but before benralizumab treatment. In some embodiments, FEV₁ can be measured pre- or post-BD administration, but after treatment with benralizumab. Thus, as used herein, “baseline % predicted FEV₁” refers to FEV₁ as measured prior to administration of benralizumab (pre- or post-BD). As used herein, “maximal post-BD % predicted FEV₁” refers to FEV₁ as measured post-BD (before or after administration of benralizumab).

As used herein, “forced vital capacity (FVC)” refers to the volume delivered during an expiration made as forcefully and completely as possible starting from full inspiration. FEV₁ and FVC values described herein are exemplary measures for assessing lung function in a patient with asthma. As described herein, FVC can be measured at baseline, e.g., baseline values determined pre- or post- bronchodilator (BD) administration, but before benralizumab treatment. In some embodiments, FVC can be measured pre- or post-BD administration, but after treatment with benralizumab. Thus, as used herein, “baseline % predicted FVC” refers to FVC as measured prior to administration of benralizumab (pre- or post-BD). As used herein, “maximal post-BD % predicted FVC” refers to FVC as measured after BD (before or after administration of benralizumab).

As used herein, an asthma “exacerbation” refers to an acute or subacute episode of progressive worsening of symptoms of asthma, including shortness of breath, wheezing, cough, and chest tightness. An “annual exacerbation rate” or “AER” refers to the number of times per year that a patient experiences asthma exacerbations.

As used herein, the “Asthma Control Questionnaire” or “ACQ” refers to a patient-reported questionnaire assessing asthma symptoms (night-time waking, symptoms on waking, activity limitation, shortness of breath, wheezing) and daily rescue bronchodilator (BD) use and FEV₁. See, Juniper et al., Eur. Respir. J 14:902-907 (1999) and Juniper et al., Chest 115:1265-1270 (1999). Questions are weighted equally and scored from 0 (totally controlled) to 6 (severely uncontrolled). Mean scores of ≤0.75 indicate well-controlled asthma; scores between 0.75 and ≤1.5 indicate partly controlled asthma; and a score >1.5 indicates uncontrolled asthma. Juniper et al., Respir. Med. 100:616-621 (2006). Individual changes of at least 0.5 are considered to be clinically meaningful. Juniper et al., Respir. Med. 99:553-558 (2005). The “ACQ-6” is a shortened version of the ACQ that assesses asthma symptoms (night-time waking, symptoms on waking, activity limitation, shortness of breath, wheezing, and short-acting β₂ agonist use) omitting the FEV₁ measurement from the original ACQ score.

Provided herein are methods for treating subjects with late-onset asthma by administering an effective amount of benralizumab or an antigen-binding fragment thereof. Also provided herein are methods for treating subjects with asthma that fall within certain asthma clinical phenotypes, e.g., cluster 3 and cluster 5 of the Severe Asthma Research Program (SARP) (Moore, W. C. et al., Am J Respir Crit Care Med. 181:315-323 (2010) by administering an effective amount of benralizumab or an antigen-binding fragment thereof. As described herein, in certain aspects, benralizumab or an antigen-binding fragment thereof is administered to the patient concurrently with additional asthma therapies.

### Benralizumab and Its Administration

Benralizumab is a humanized, afucosylated, monoclonal antibody (mAb) that targets the alpha chain of the interleukin-5 receptor (IL-5Rα). See, Kolbeck, R. et al., J Allergy Clin. Immunol. 125:1344-1353 (2010). Benralizumab exerts its effect by inducing the direct, rapid, and nearly complete depletion of blood eosinophils though enhanced antibody-dependent cell-mediated cytotoxicity, an apoptotic process of eosinophil elimination involving natural killer cells. Id.; Pham, T. H. et al., Resp. Med. 111:21-29 (2016). Airway eosinophils (tissue and sputum) are also extensively depleted. Laviolette, M. et al., J Allergy Clin. Immunol. 132:1086-1096 (2013).

Information regarding benralizumab (or antibody-binding fragments thereof) for use in the methods provided herein can be found in U.S. Patent Appl. Publ. No. US 2010/0291073, U.S. Pat. Nos. 7,718,175, 7,179,464, 8,101,185, and U.S. Patent Appl. Publ. No. US 2019/0201535, the disclosure of which is incorporated herein by reference in its entirety. Benralizumab and antigen-binding fragments thereof for use in the methods provided herein comprise a heavy chain and a light chain or a heavy chain variable region and a light chain variable region.

In a further aspect, benralizumab or an antigen-binding fragment thereof for use in the methods provided herein includes any one of the amino acid sequences of SEQ ID NOs: 1-4. In a specific aspect, benralizumab or an antigen-binding fragment thereof for use in the methods provided herein comprises a light chain variable region comprising the amino acid sequence of SEQ ID NO:1 and a heavy chain variable region comprising the amino acid sequence of SEQ ID NO:3.

In a specific aspect, benralizumab or an antigen-binding fragment thereof for use in the methods provided herein comprises a light chain comprising the amino acid sequence of SEQ ID NO: 2 and heavy chain comprising the amino acid sequence of SEQ ID NO:4. In a specific aspect, benralizumab or an antigen-binding fragment thereof for use in the methods provided herein comprises a heavy chain variable region and a light chain variable region, wherein the heavy chain variable region comprises the Kabat-defined CDR1, CDR2, and CDR3 sequences of SEQ ID NOs: 7-9, and wherein the light chain variable region comprises the Kabat-defined CDR1, CDR2, and CDR3 sequences of SEQ ID NOs: 10-12. Those of ordinary skill in the art would easily be able to identify Chothia-defined, Abm-defined or other CDRs. In a specific aspect, benralizumab or an antigen-binding fragment thereof for use in the methods provided herein comprises the variable heavy chain and variable light chain CDR sequences of the KM1259 antibody as disclosed in U.S. Pat. No. 6,018,032, which is herein incorporated by reference in its entirety.

Two Phase III clinical trials, SIROCCO (ClinicalTrials.gov identifier NCT01928771) and CALIMA (ClinicalTrials.gov identifier NCT01914757), demonstrated that benralizumab, in combination with high-dosage inhaled corticosteroids/long-acting β2-agonists (ICS/LABA), significantly reduced asthma exacerbations and improved lung function and disease control for patients with severe, uncontrolled asthma and blood eosinophil counts ≥300 cells/μL versus placebo. See, Bleeker, E. R. et al., Lancet 388:2115-2127 (2016)) and FitzGerald, J. M. et al., Lancet 388:2128-2141 (2016), respectively. A 30 mg subcutaneous formulation of benralizumab, administered once every 8 weeks (Q8W), with the first three doses administered once every 4 weeks (Q4W), has subsequently been approved in several markets as add-on maintenance treatment for patients with severe, uncontrolled eosinophilic asthma. See, AstraZeneca. Fasenra™ (Benralizumab), Prescribing Information (2017), www.azpicentral.com/fasenra/fasenra_pi.pdf, date last updated: November, 2017; date last accessed: Jan. 10, 2018; AstraZeneca. Fasenra™ (Benralizumab), Summary of Product Characteristics (2018), http://ec.europa.eu/health/documents/community-register/2018/20180108139598/anx_139598_en.pdf; date last accessed: Mar. 13, 2018.

In certain aspects, a patient presenting at a physician's office or ED with asthma is administered benralizumab or an antigen-binding fragment thereof. Given the ability of benralizumab to reduce or deplete eosinophil counts for up to 12 weeks or more (see US 2010/0291073), benralizumab or an antigen-binding fragment thereof can be administered only once or infrequently while still providing benefit to the patient in reducing asthma symptoms.

In further aspects the patient is administered additional follow-on doses. Follow-on doses can be administered at various time intervals depending on the patient's age, weight, ability to comply with physician instructions, clinical assessment, eosinophil count (blood or sputum eosinophils), Eosinophilic Cationic Protein (ECP) measurement, Eosinophil-derived neurotoxin measurement (EDN), Major Basic Protein (MBP) measurement and other factors, including the judgment of the attending physician.

The intervals between doses of benralizumab can be every 4 weeks, every 5 weeks, every 6 weeks, every 8 weeks, every 10 weeks, every 12 weeks, or longer intervals. In certain aspects the intervals between doses can be every 4 weeks, every 8 weeks, or every 12 weeks. In certain aspects, the single dose or first dose is administered to the asthma patient shortly after the patient presents with an exacerbation, e.g., a mild, moderate or severe exacerbation. For example, benralizumab or an antigen-binding fragment thereof can be administered during a presenting clinic or hospital visit, or in the case of very severe exacerbations, within 1, 2, 3, 4, 5, 6, 7, or more days, e.g., 7 days of the acute exacerbation, allowing the patient's symptoms to stabilize prior to administration of benralizumab.

In some embodiments, at least two doses of benralizumab or an antigen-binding fragment thereof are administered to the patient. In some embodiments, at least three doses, at least four doses, at least five doses, at least six doses, or at least seven doses are administered to the patient. In some embodiments, benralizumab or an antigen-binding fragment thereof is administered over the course of four weeks, over the course of eight weeks, over the course of twelve weeks, over the course of twenty-four weeks, or over the course of a year.

The amount of benralizumab or antigen-binding fragment thereof to be administered to the patient will depend on various parameters such as the patient's age, weight, clinical assessment, eosinophil count (blood or sputum eosinophils), Eosinophilic Cationic Protein (ECP) measurement, Eosinophil-derived neurotoxin measurement (EDN), Major Basic Protein (MBP) measurement and other factors, including the judgment of the attending physician. In certain aspects, the dosage or dosage interval is not dependent on the eosinophil level.

In certain aspects the patient is administered one or more doses of benralizumab or an antigen-binding fragment thereof, wherein the dose is about 2 mg to about 100 mg, for example about 20 mg to about 100 mg, or about 30 mg to about 100 mg. In certain specific aspects, the patient is administered one or more doses of benralizumab or an antigen-binding fragment thereof where the dose is about 20 mg, about 30 mg, about 40 mg, about 50 mg, about 60 mg, about 70 mg, about 80 mg, about 90 mg, or about 100 mg.

In certain aspects, administration of benralizumab or an antigen-binding fragment thereof according to the methods provided herein is through parenteral administration. In some embodiments, benralizumab or an antigen-binding fragment thereof is administered by subcutaneous injection into, e.g., the upper arm, thigh, or abdomen.

In a preferred embodiment, the dose of benralizumab is about 30 mg administered once every 4 weeks. In a preferred embodiment, the dose of benralizumab is about 30 mg administered once every 4 weeks for the first three doses, and then once every 8 weeks thereafter by subcutaneous injection.

### Additional Asthma Therapies

In certain aspects, benralizumab or an antigen-binding fragment thereof is administered according to the methods provided herein in combination with or in conjunction with additional asthma therapies. Such additional asthma therapies include, without limitation, corticosteroid therapy, long-term or short-term bronchodilator (BD) treatment, long-acting β₂ agonist (LABA) treatment, short-acting β₂ agonist (SABA) treatment, oxygen supplementation, or other standard therapies as described, e.g., in the National Asthma Education and Prevention Program (NAEPP) Guidelines. In some embodiments, the patient may have used additional asthma therapies, as described herein, prior to administration of benralizumab or an antigen-binding fragment thereof.

As used herein, “corticosteroid therapy” includes inhaled corticosteroid (ICS) therapy (including high-dosage ICS), oral corticosteroids, and systemic corticosteroids.

In certain aspects, the asthma patient was prescribed or has been using a medium-dose of inhaled corticosteroids (ICS) use prior to the administration of benralizumab or an antigen-binding fragment thereof. In certain aspects, the asthma patient concurrently uses a medium-dose of ICS with benralizumab or an antigen-binding fragment thereof. A medium-dose of ICS can be a dose of at least 600 μg to 1,200 μg budesonide daily or an equivalent dose of another ICS.

In certain aspects, the asthma patient was prescribed or has been using a high-dose of ICS use prior to the administration of benralizumab or an antigen-binding fragment thereof. In certain aspects, the asthma patient concurrently uses a high-dose of ICS with benralizumab or an antigen-binding fragment thereof. A high-dose of ICS can be a dose of at least 1,200 μg budesonide daily or an equivalent dose of another ICS. A high dose of ICS can also be a dose of greater than 1,200 μg to 2000 μg budesonide daily or an equivalent dose of another ICS.

In certain aspects, the asthma patient was prescribed or has been using oral corticosteroids prior to the administration of benralizumab or an antigen-binding fragment thereof. In certain aspects, the asthma patient concurrently uses oral corticosteroids with benralizumab or an antigen-binding fragment thereof. In certain aspects, administration of benralizumab or an antigen-binding fragment thereof decreases the use of oral corticosteroids in an asthma patient. In certain aspects, the administration decreases the use of oral corticosteroids in an asthma patient by at least 50%.

In certain aspects, the asthma patient was prescribed or has been using a long-acting β₂ agonist (LABA) prior to the administration of benralizumab or an antigen-binding fragment thereof. In certain aspects, the asthma patient concurrently uses a LABA with benralizumab or an antigen-binding fragment thereof.

In certain aspects, the asthma patient was prescribed or has been using a short-acting β₂ agonist (SABA) prior to the administration of benralizumab or an antigen-binding fragment thereof. In certain aspects, the asthma patient concurrently uses a SABA with benralizumab or an antigen-binding fragment thereof.

In certain aspects, the asthma patient was prescribed or has been using both ICS and LABA prior to the administration of benralizumab or an antigen-binding fragment thereof.

In certain aspects, the asthma patient concurrently uses both ICS and LABA with benralizumab or an antigen-binding fragment thereof.

### Determination of Asthma Phenotype Using SARP Variables

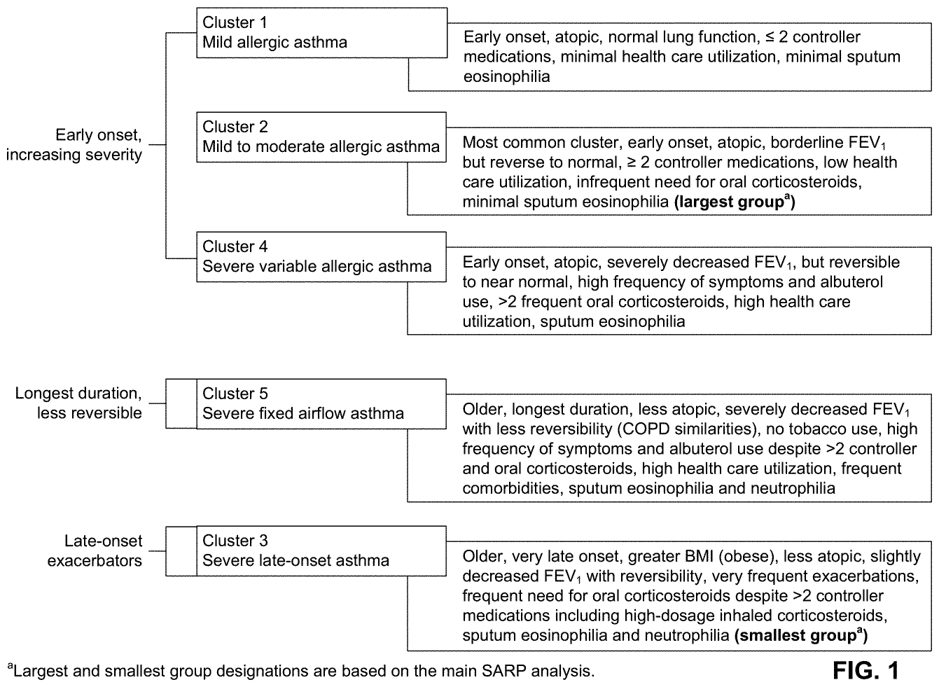

In certain aspects, prior to administration of benralizumab or an antigen-binding fragment thereof to a patient with asthma, the clinical asthma phenotype of the patient is determined using 3, 4, 5, 6, 7, 8, 9, 10, or 11 clinical characteristics (a.k.a. variables) that were identified in the cluster analysis of 726 asthma subjects in the National Heart, Lung, and Blood Institute's Severe Asthma Research Program (SARP). See, Moore, W. C. et al., Am J Respir Crit Care Med. 181:315-323 (2010). SARP identified five distinct clinical phenotypes of asthma (i.e., Cluster 1, 2, 3, 4, and 5) and FIG. 1 provides the characteristics for each cluster.

In some embodiments, prior to administration of benralizumab or an antigen-binding fragment thereof to a patient with asthma, any or all of the following 11 clinical characteristics were assessed: age at asthma onset; forced expiratory volume in 1 second (FEV₁); forced vital capacity (FVC); FEV₁/FVC; maximal post-bronchodilator (BD) FEV₁; maximal post-BD FVC; percentage change in post-BD FEV₁; asthma duration; gender of the patient; frequency of β2-agonist use; and high-dosage inhaled corticosteroid (ICS) dosage. Such phenotypic characterization can be assessed using e.g., comprehensive questionnaires (to assess, e.g., demographics, frequency of asthma symptoms, age of asthma onset, asthma duration, dosage and frequency of asthma medications, such as β₂ agonists and corticosteroids); pulmonary function testing (e.g., pre-BD FEV₁ and FVC, and post-BD FEV₁ and FVC); and a determination of the number of asthma exacerbations in the last 12 months.

In some embodiments, prior to administration of benralizumab or an antigen-binding fragment thereof to a patient with asthma, the following 3 clinical characteristics were assessed: age at asthma onset, pre-BD FEV₁, and maximal post-BD FEV₁.

In some embodiments, the patient's age of asthma onset is 16 years of age or older, which is defined herein as “late-onset asthma.” In some embodiments, the patient's age of asthma onset ranges from 16 to 56 years old. In some embodiments, the patient's age of asthma onset is 47±9.4 years. In some embodiments, the patient's age of asthma onset is 33±17.0 years.

In some embodiments, the patient's baseline pre-BD FEV₁ is 66±7.7%. In some embodiments, the patient's baseline pre-BD FEV₁ is 43±9.4.

In some embodiments, the patient's maximal post-BD FEV₁ is 78±12%. In some embodiments, the patient's maximal post-BD FEV₁ is 56±15%.

In some embodiments, the patient's age of asthma onset is 47±9.4 years, baseline pre-BD FEV₁ is 66±7.7%, and maximal post-BD FEV₁ is 78±12%. Patients with this phenotypic characterization would fall within SARP clinical cluster 3.

In some embodiments, the patient's age of asthma onset is 33±17.0 years, baseline pre-BD FEV₁ is 43±9.4%, and maximal post-BD FEV₁ is 56±15%. Patients with this phenotypic characterization would fall within SARP clinical cluster 5.

### Methods for Treating Patients in SARP Clinical Cluster 3 or Clinical Cluster 5 with Benralizumab or an Antigen-Binding Fragment Thereof

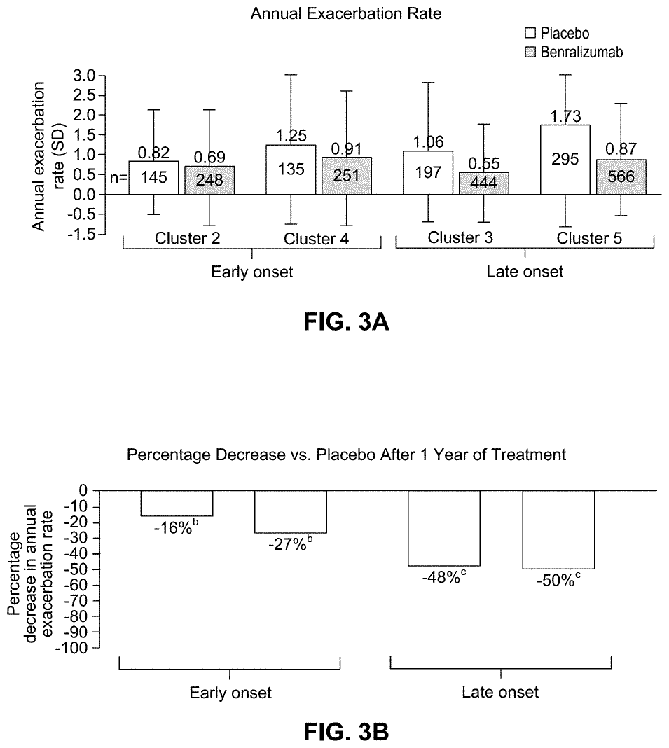

As described in Example 1, and shown in Table 3 and FIGS. 3A and 3B, while patients in all clusters had AER reductions following treatment with benralizumab vs. placebo, patients in SARP clinical cluster 3 or clinical cluster 5 (i.e., late-onset asthma clusters) had the greatest reductions in AER, with cluster 3 showing an AER reduction of −48% compared to placebo and cluster 5 showing an AER reduction of −50% compared to placebo. In addition, AER reductions in clusters 3 and 5, were even more pronounced when the patient's blood eosinophil level was ≥300 cells/4., showing an AER reduction of −57% compared to placebo for cluster 3 patients and an AER reduction of −53% compared to placebo for cluster 5 patients. See, Table 4.

Thus, in some embodiments, administration of benralizumab or an antigen-binding fragment thereof reduces AER by at least 45% (e.g., 45% to compared to a patient not administered the benralizumab or antigen-binding fragment thereof. In some embodiments, administration of benralizumab or an antigen-binding fragment thereof reduces AER by at least 50% compared to a patient not administered the benralizumab or antigen-binding fragment thereof.

In some embodiments, administration of benralizumab or an antigen-binding fragment thereof reduces a patient's AER as compared to the patient's AER prior to the administration. In some embodiments, administration of benralizumab or an antigen-binding fragment thereof reduces a patient's AER by at least 0.5.

In some embodiments, administration of benralizumab or an antigen-binding fragment thereof reduces a patient's AER by at least 1.0.

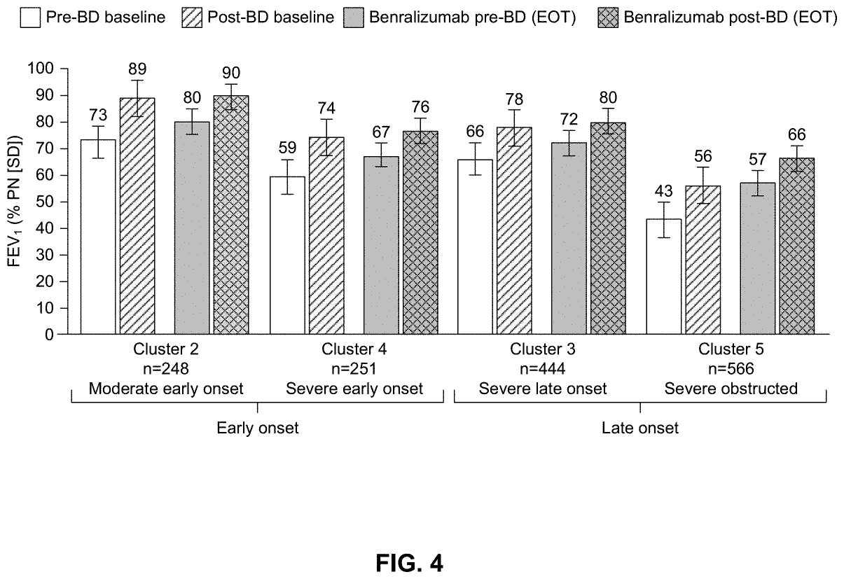

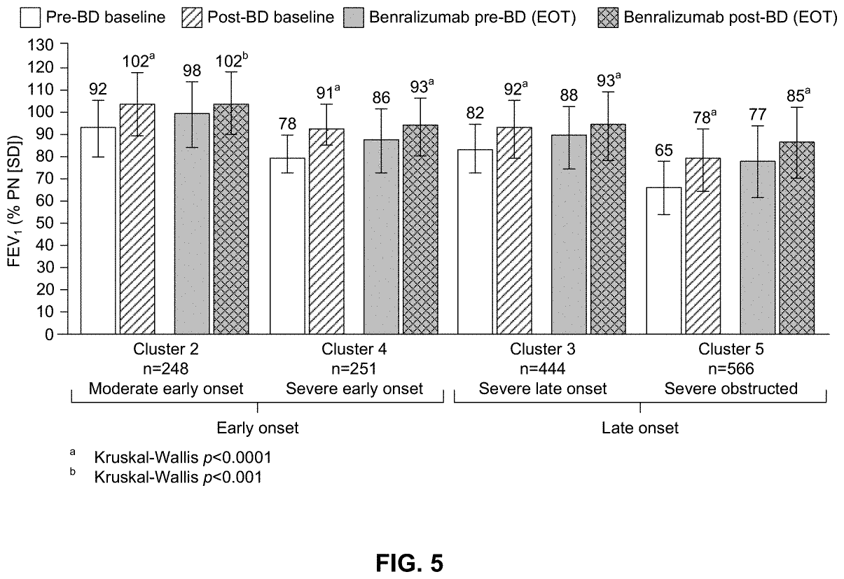

Also, as described in Example 1, and shown in FIG. 4 (FEV₁) and FIG. 5 (FVC), lung function improvements were greater with benzalizumab vs. placebo in clusters 3, 4, and 5 while maintaining an effect on post-BD response. Cluster 3 (+130 mL; p=0.0005) and Cluster 5 (+160 mL; p<0.0001) had the greatest improvements in FEV₁ with benralizumab vs. placebo from baseline. Cluster 5 FEV₁ increased 410 mL vs. baseline (p<0.0001).

Thus, in some embodiments, administration of benralizumab or an antigen-binding fragment thereof improves pre-bronchodilator (BD) FEV₁. In some embodiments, the pre-BD FEV₁ is increased by at least 6% (e.g., 6% to 30%, 6% to 25%, 6% to 20%, or 6% to 15%). In some embodiments, the pre-BD FEV₁ is increased by at least 14% (e.g., 14% to 30%, 14% to 25%, or 14% to 20%).

In some embodiments, administration of benralizumab or an antigen-binding fragment thereof improves post-bronchodilator (BD) FEV₁. In some embodiments, the post-BD FEV₁ is increased by at least 2% (e.g., 2% to 25%, 2% to 20%, 2% to 15%, 2% to 10% or 2% to 5). In some embodiments, the post-BD FEV₁ is increased by at least 10% (e.g., 10% to 25%, 10% to 20%, or 10% to 15%).

The methods provided herein can significantly reduce exacerbations of asthma. Reduction can be measured based on the expected exacerbations predicted based on a large patient population, or based on the individual patient's history of exacerbations. In certain aspects, the patient population is those patients who had ≥2 exacerbations requiring systemic corticosteroid bursts in the past year. In certain aspects, the patient population is those patients who had ≥2 exacerbations requiring systemic corticosteroid bursts in the past year and ≤6 exacerbations requiring systemic corticosteroid bursts in the past year. In certain aspects, the patient population is patients having an eosinophil count of at least 300 cells/μl.

In certain aspects, use of the methods provided herein, i.e., administration of benralizumab or an antigen-binding fragment thereof, reduces the number of exacerbations experienced by the patient over a 24-week period following administration of benralizumab or an antigen-binding fragment thereof, as compared to the number of exacerbations expected according to the patient's history, as compared to the average number of exacerbations expected in a comparable population of patients, or as compared to a comparable population treated with placebo over the same time period. In certain aspects, the patient can receive follow on doses of benralizumab or an antigen-binding fragment thereof at periodic intervals, e.g., every 4 weeks, every 5 weeks, every 6 weeks, every 8 weeks, every 12 weeks, or as scheduled based on patient's age, weight, ability to comply with physician instructions, clinical assessment, eosinophil count (blood or sputum eosinophils), Eosinophilic Cationic Protein (ECP) measurement, Eosinophil-derived neurotoxin measurement (EDN), Major Basic Protein (MBP) measurement and other factors, including the judgment of the attending physician. Use of the methods provided herein can reduce the frequency of exacerbations by 10%, 20%, 30%, 35%, 40%, 45%, 50%, 55%, 60%, 65%, 70%, 75%, 80%, 85%, 90%, 95% or 100% over the 24-week period.

In other aspects, use of the methods provided herein, i.e., administration of benralizumab or an antigen-binding fragment thereof to an asthma patient, reduces the number of exacerbations experienced by the patient over a 52-week period (i.e., the annual exacerbation rate) following administration of benralizumab or an antigen-binding fragment thereof, as compared to the number of exacerbations expected according to the patient's history, as compared to the average number of exacerbations expected in a comparable population of patients, or as compared to a comparable population treated with placebo over the same time period. In certain aspects, the patient can receive follow on doses of benralizumab or an antigen-binding fragment thereof at periodic intervals, e.g., every 4 weeks, every 5 weeks, every 6 weeks, every 8 weeks, every 12 weeks, or as scheduled based on patient's age, weight, ability to comply with physician instructions, clinical assessment, eosinophil count (blood or sputum eosinophils), Eosinophilic Cationic Protein (ECP) measurement, Eosinophil-derived neurotoxin measurement (EDN), Major Basic Protein (MBP) measurement and other factors, including the judgment of the attending physician. In certain aspects, the interval is every 4 weeks, every 8 weeks or every 12 weeks. Use of the methods provided herein can reduce the annual exacerbations by 10%, 20%, 30%, 35%, 40%, 45%, 50%, 55%, 60%, 65%, 70%, 75% , 80%, 85%, 90%, 95% or 100%.

In certain aspects, use of the methods provided herein, i.e., administration of benralizumab or an antigen-binding fragment thereof to an asthma patient (e.g. a patient with late-onset asthma, as described herein), reduces the annual exacerbation rate, increases forced expiratory volume (FEV₁), and/or improves an asthma questionnaire score (e.g., the asthma control questionnaire (ACQ)).

In certain aspects, the patient is “eosinophilic positive” meaning the patient is one whose asthma is likely to be eosinophilic.

In certain aspects, the asthma patient (e.g. a patient with late-onset asthma, as described herein), has a particular blood eosinophil count, e.g., prior to the administration of benralizumab or an antigen-binding fragment thereof. Blood eosinophil counts can be measured, for example, using a complete blood count (CBC) with cell differential.

In certain aspects, the asthma patient has a blood eosinophil count of at least 300 cells/μl prior to the administration of benralizumab or an antigen-binding fragment thereof. In certain aspects, the asthma patient has a blood eosinophil count of greater than or equal to (≥) 300 cells/μl prior to the administration of benralizumab or an antigen-binding fragment thereof. In certain aspects, the asthma patient has a blood eosinophil count of, at least 350 cells/μl, at least 400 cells/μl, at least 450 cells/μl, or at least 500 cells/μl prior to the administration of benralizumab or an antigen-binding fragment thereof.

In certain aspects, the asthma patient has a blood eosinophil count of less than 300 cells/μl prior to the administration of benralizumab or an antigen-binding fragment thereof. In certain aspects, the asthma patient has a blood eosinophil count of at least 100 cells/μl, at least 150 cells/μl, at least 180 cells/μl, at least 200 cells/μl, or at least 250 cells/μl prior to the administration of benralizumab or an antigen-binding fragment thereof.

In certain aspects, the asthma patient has a blood eosinophil count of at least 300 cells/μl and high ICS use prior to the administration of benralizumab or an antigen-binding fragment thereof.

In certain aspects, the asthma patient (e.g. a patient with late-onset asthma, as described herein) has a forced expiratory volume in 1 second (FEV₁) of at least 40% and less than 90% predicted value prior to the administration of benralizumab or an antigen-binding fragment thereof. In some embodiments, the FEV₁ was greater than 70% predicted value prior to the administration of benralizumab or an antigen-binding fragment thereof. In some embodiments, the FEV₁ was greater than 70% and less than 90% predicted value prior to the administration of benralizumab or an antigen-binding fragment thereof. In some embodiments, the FEV₁ was at least 75% predicted value prior to the administration of benralizumab or an antigen-binding fragment thereof. In some embodiments, the FEV₁ was at least 75% and less than 90% prior predicted value to the administration of benralizumab or an antigen-binding fragment thereof. In some embodiments, the FEV₁ was at least 80% predicted value prior to the administration of benralizumab or an antigen-binding fragment thereof. In some embodiments, the FEV₁ was at least 80% and less than 90% predicted value prior to the administration of benralizumab or an antigen-binding fragment thereof.

### Predicting an Enhanced Response to Benralizumab or an Antigen-Binding Fragment Thereof

In one aspect, the present disclosure provides a method of predicting an asthma patient's therapeutic response to benralizumab or an antigen-binding fragment thereof, the method comprising, determining, prior to administration of the benralizumab or antigen-binding fragment thereof, the SARP clinical cluster of the asthma, as described herein.

In some embodiments, the method comprises predicting an enhanced response to the benralizumab or antigen-binding fragment thereof if the SARP clinical cluster is determined to be cluster 3 or cluster 5.

In some embodiments, the method further comprises administering the benralizumab or antigen-binding fragment thereof to the patient if the SARP clinical cluster of the patient's asthma is determined to be cluster 3 or cluster 5.

The following example further illustrates the invention but, of course, should not be construed as in any way limiting its scope.

### EXAMPLE 1

### Background

Asthma is a heterogeneous disease with that can be categorized into different phenotypes by distinct clusters of demographic, clinical, and/or pathophysiological characteristics. In 2010, the National Heart, Lung, and Blood Institute Severe Asthma Research Program (SARP) identified five distinct clinical phenotypes of asthma (i.e., Cluster 1, 2, 3, 4, and 5) using an unsupervised hierarchical cluster analysis of 726 subjects. See, Moore, W. C. et al., Am J Respir Crit Care Med. 181:315-323 (2010).

In the SIROCCO (Bleeker, E. R. et al., Lancet 388:2115-2127 (2016)) and CALIMA (FitzGerald, J. M. et al., Lancet 388:2128-2141 (2016)) Phase III clinical trials, benralizumab significantly reduced asthma exacerbations and improved lung function for patients with severe, uncontrolled asthma with baseline blood eosinophil counts ≥300 cells/μL receiving high-dosage inhaled corticosteroids (ICS)/long-acting β2-agonists. In this example, patients randomized in the SIROCCO/CALIMA studies were characterized through SARP clinical clusters (see FIG. 1) to evaluate the potential for subphenotypes of patients with differential treatment effects for benralizumab compared with placebo.

### Methods

Data were pooled for patients (N=2,281) from the 48-week SIROCCO (n=1,082) (Bleeker, E. R. et al., Lancet 388:2115-2127 (2016) and 56-week CALIMA (n=1,199) studies. (FitzGerald, J. M. et al., Lancet 388:2128-2141 (2016). In these studies, recruitment aimed to enroll patients with blood eosinophil counts ≥300 cells/μL and <300 cells/μL at a ratio of approximately 2:1, respectively. Patients were randomized to receive benralizumab 30 mg subcutaneously either every 4 weeks (Q4W) or every 8 weeks (first three doses Q4W) or placebo. The data were combined for benralizumab treatment arms in these analyses.

Patients were assigned to SARP clinical clusters through the discriminant function, which uses 11 clinical characteristics: forced expiratory volume in 1 second (FEV₁); forced vital capacity (FVC); FEV₁/FVC; maximal post-bronchodilator (BD) FEV₁; maximal post-BD FVC; percentage change in post-BD FEV₁; age at asthma onset; asthma duration; sex; frequency of β2-agonist use; and high dosage inhaled corticosteroid (ICS) dosage. See, Moore, W. C. et al., Am J Respir Crit Care Med. 181:315-323 (2010). Baseline clinical characteristics and responses across clusters, treatment groups, and baseline blood eosinophil counts (<300 cells/μL and ≥300 cells/μL) were compared. In addition, exacerbation rate, exacerbation rate reduction, and lung function measures were analyzed with the Kruskal-Wallis test (nominal p-values).

### Results

The demographics and baseline clinical characteristics for the pooled SIROCCO/CALIMA patients across all blood eosinophil counts are provided in Table 1.

TABLE 1
Demographics and Baseline Clinical Characteristics
of Patients Enrolled in SIROCCO/CALIMA Phase
III Studies (All Eosinophil Counts)

| | SIROCCO/CALIMA |
|---|---|
| | (N = 2,281) |
| Age at asthma onset [years] | 30 ± 19 |
| Asthma duration [years] | 20 ± 15 |
| Sex (female), % | 63 |
| Baseline % predicted FEV₁ | 57 ± 15 |
| Baseline % predicted FVC | 77 ± 16 |
| Baseline FEV₁/FVC | 0.61 ± 0.13 |
| Maximal % predicted FEV₁ | 71 ± 18 |
| Maximal % predicted FVC | 88 ± 16 |
| FEV₁ reversibility | 26 ± 24 |
| Steroid use, %^{a} | |
| 0 | 0 |
| 1 | 34 |
| 2 | 54 |
| 3 | 12 |
| SABA use, %^{b} | |
| 0 | 12 |
| 1 | 41 |
| 2 | 40 |
| 3 | 7 |
| 4 | 0 |
| BMI [kg/m^{2]} | 29 ± 6.7 |

Data are mean ± SD unless otherwise noted.
^{a}0 = none, 1 = low- to medium-dosage ICS, 2 = high-dosage ICS, 3 = systemic corticosteroids.
^{b}0 = none, 1 = once monthly, 2 = once weekly, 3 = once daily, 4 = more than once daily. SABA = short-acting β₂-agonist.

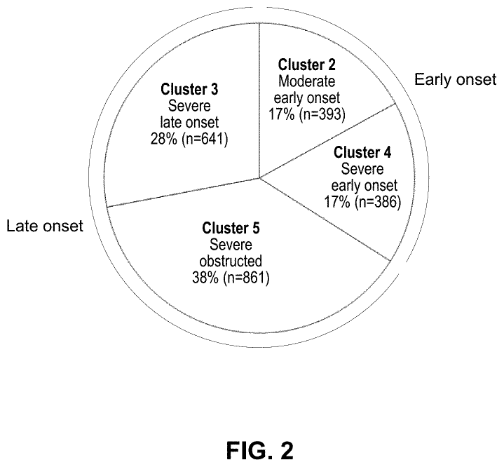

Patients from SIROCCO/CALIMA were grouped into four of five SARP clusters. See, FIG. 2. 17% of patients were grouped into Cluster 2 (n=393). Of these, 81% were atopic by Phadiatop test, with a mean age at asthma onset of 15 years. 28% of patients were grouped into Cluster 3 (n=641). Of these, 50% were atopic by Phadiatop test, with a mean age at asthma onset of 47 years. 17% of patients were grouped into Cluster 4 (n=386). Of these, 82% were atopic by Phadiatop test, with a mean age at asthma onset of 11 years. 38% of patients were grouped into Cluster 5 (n=861). Of these, 55% were atopic by Phadiatop test, with persistent airflow obstruction and a mean age at asthma onset of 33 years. These clusters constitute distinct patient subpopulations with differing demographics and baseline clinical characteristics (see Table 2).

TABLE 2
Demographics and Baseline Clinical Characteristics of SIROCCO/CALIMA
Patients by Assigned SARP Cluster (All Eosinophil Counts)

| | Early onset | | Late onset | |
|---|---|---|---|---|
| | Cluster 2 | Cluster 4 | Cluster 3 | Cluster 5 |
| | Moderate | Severe | Severe | Severe |
| | early onset | early onset | late onset | obstructed |
| | (n = 393) | (n = 386) | (n = 641) | (n = 861) |
| Age at asthma onset [years] | 15 ± 12.0 | 11 ± 9.4 | 47 ± 9.4 | 33 ± 17.0 |
| Asthma duration [years] | 19 ± 12 | 33 ± 15 | 10 ± 7 | 21 ± 15 |
| Sex (female), % | 63 | 56 | 63 | 66 |
| Baseline % predicted FEV₁ | 73 ±7.4 | 59 ± 7.3 | 66 ± 7.7 | 43 ± 9.4 |
| Baseline % predicted FVC | 92 ± 11 | 78 ± 10 | 82 ± 11 | 65 ± 12 |
| Baseline FEV₁/FVC | 0.67 ± 0.11 | 0.63 ± 0.12 | 0.65 ± 0.10 | 0.54 ± 0.12 |
| Post-BD % predicted FEV₁ | 89 ± 13 | 74 ± 12 | 78 ± 12 | 56 ± 15 |
| Post-BD % predicted FVC | 102 ± 13 | 90 ± 12 | 91 ± 14 | 77 ± 15 |
| FEV₁ reversibility | 23 ± 18 | 26 ± 20 | 18 ± 15 | 32 ± 30 |
| Steroid use, %^{a} | | | | |
| 0 | 0 | 0 | 0 | 0 |
| 1 | 47 | 36 | 32 | 28 |
| 2 | 50 | 54 | 58 | 53 |
| 3 | 3 | 10 | 10 | 19 |
| SABA use, %^{b} | | | | |
| 0 | 14 | 9 | 16 | 10 |
| 1 | 47 | 42 | 44 | 35 |
| 2 | 35 | 42 | 34 | 45 |
| 3 | 4 | 7 | 6 | 9 |
| 4 | 0 | 0 | 0 | 0 |
| BMI [kg/m²] | 26 ± 6.2 | 30 ± 7.9 | 29 ± 5.6 | 29 ± 6.7 |
| Nasal polyposis (yes), % | 10 | 13 | 20 | 21 |
| Allergic rhinitis (yes), % | 72 | 63 | 49 | 50 |
| Atopicc (yes), % | 81 | 82 | 50 | 55 |
| Blood eosinophil count | 482 ± 403 | 448 ± 348 | 471 ± 371 | 489 ± 412 |
| [cells/μL] | | | | |
| ACQ-6 score (baseline) | 2.5 ± 0.86 | 2.7 ± 0.91 | 2.6 ± 0.87 | 2.9 ± 0.95 |
| Number of exacerbations | 2.7 ± 1.6 | 2.7 ± 1.7 | 2.6 ± 1.6 | 3.0 ± 1.8 |
| (in the last 12 months) | | | | |

Data are mean ± SD unless otherwise noted. P-values were derived with the Kruskal-Wallis test for quantitative variables and the chi-square test for categorical variables.
^{a}0 = none, 1 = low- to medium-dosage ICS, 2 = high-dosage ICS, 3 = systemic corticosteroids.
^{b}0 = none, 1 = once monthly, 2 = once weekly, 3 = once daily, 4 = more than once daily.
^{c} Atopic by Phadiatop test.

Pooled patients across blood eosinophil counts in all clusters had reductions in annual exacerbation rates with benralizumab vs. placebo (see FIG. 3 and Table 3).

TABLE 3
Effects of Benralizumab (Benra) on Annual Exacerbation
Rate (AER) and Lung Function vs. Placebo by
SARP Cluster (All Eosinophil Counts)

| | Cluster 2: Moderate early onset (n = 393) | | |
|---|---|---|---|
| | Placebo | Benra^{a} | |
| | (n = 48) | (n = 79) | P-value |
| Annual exacerbation rate | 0.82 ± 1.32 | 0.69 ± 1.46 | 0.18 |
| ΔFEV₁ from baseline [L] | 0.28 ± 0.48 | 0.25 ± 0.49 | 0.29 |
| ΔFVC from baseline [L] | 0.05 ± 0.38 | 0.01 ± 0.4 | 0.46 |
| | Cluster 4: Severe early onset (n = 386) | | |
| | Placebo | Benra^{a} | |
| | (n = 135) | (n = 251) | P-value |
| Annual exacerbation rate | 1.25 ± 1.98 | 0.91 ± 1.68 | 0.17 |
| ΔFEV₁ from baseline [L] | 0.22 ± 0.47 | 0.28 ± 0.52 | 0.45 |
| ΔFVC from baseline [L] | 0.06 ± 0.42 | 0.07 ± 0.39 | 0.71 |

| | Cluster 3: Severe late onset (n = 641) | | |
|---|---|---|---|
| | Placebo | Benra^{a} | |
| | (n = 197) | (n = 444) | P-value |
| Annual exacerbation rate | 1.06 ± 1.73 | 0.55 ± 1.23 | <0.0001 |
| ΔFEV₁ from baseline [L] | 0.04 ± 0.4 | 0.17 ± 0.38 | 0.0005 |
| ΔFVC from baseline [L] | 0.02 ± 0.45 | 0.05 ± 0.35 | 0.47 |

| | Cluster 5: Severe obstructed (n = 861) | | |
|---|---|---|---|
| | Placebo | Benra^{a} | |
| | (n = 295) | (n = 444) | P-value |
| Annual exacerbation rate | 1.73 ± 2.56 | 0.87 ± 1.43 | <0.0001 |
| ΔFEV₁ from baseline [L] | 0.25 ± 0.48 | 0.41 ± 0.52 | <0.0001 |
| ΔFVC from baseline [L] | 0.13 ± 0.45 | 0.27 ± 0.57 | 0.004 |

P-values for all variables were derived with the Kruskal-Wallis test.
^{a}Combined data for benralizumab 30 mg every 4 weeks (Q4W) and 30 mg every 8 weeks (first three doses Q4W) treatment groups.

Clusters associated with late-onset asthma had the greatest reductions in annual exacerbation rates. In particular, cluster 3 had an annual exacerbation rate reduction of −48% (p<0.0001) and cluster 5 had an annual exacerbation rate reduction of −50% (p<0.0001). When each cluster was further subgrouped by patients with blood eosinophil counts ≥300 cells/μL vs. <300 cells/μL, those with ≥300 cells/μL experienced more pronounced exacerbation rate reductions (Table 4).

TABLE 4
Annual Exacerbation Rate (AER) and Lung Function Improvements by SARP
Cluster and Blood Eosinophil Count for Benralizumab^{a} vs. Placebo

| | Cluster 2: Moderate early onset (n = 393) | | | | | |
|---|---|---|---|---|---|---|
| | Blood eosinophil counts | | | Blood eosinophil counts | | |
| | <300 cells/μL | | | ≥300 cells/μL | | |
| | Placebo | Benra | | Placebo | Benra | |
| | (n = 48) | (n = 79) | P-value | (n = 97) | (n = 169) | P-value |
| Annual exacerbation rate | 0.99 ± 1.4 | 0.84 ± 1.5 | 0.47 | 0.74 ± 1.3 | 0.62 ± 1.4 | 0.27 |
| ΔFEV₁ from baseline [L] | 0.22 ± 0.48 | 0.24 ± 0.53 | 0.99 | 0.31 ± 0.49 | 0.26 ± 0.47 | 0.17 |
| ΔFVC from baseline [L] | 0.03 ± 0.38 | 0.02 ± 0.34 | 0.90 | 0.05 ± 0.37 | 0.01 ± 0.43 | 0.43 |
| | Cluster 4: Severe early onset (n = 386) | | | | | |
| | Blood eosinophil counts | | | Blood eosinophil counts | | |
| | <300 cells/μL | | | ≥300 cells/μL | | |
| | Placebo | Benra | | Placebo | Benra | |
| | (n = 40) | (n = 84) | P-value | (n = 95) | (n = 167) | P-value |
| Annual exacerbation rate | 1.60 ± 2.5 | 1.10 ± 2.3 | 0.08 | 1.10 ± 1.7 | 0.83 ± 1.3 | 0.66 |
| ΔFEV₁ from baseline [L] | 0.21 ± 0.42 | 0.26 ± 0.57 | 0.94 | 0.23 ± 0.49 | 0.30 ± 0.50 | 0.35 |
| ΔFVC from baseline [L] | 0.06 ± 0.38 | 0.09 ± 0.52 | 0.91 | 0.06 ± 0.44 | 0.06 ± 0.32 | 0.57 |
| | Cluster 3: Severe late onset (n = 641) | | | | | |
| | Blood eosinophil counts | | | Blood eosinophil counts | | |
| | <300 cells/μL | | | ≥300 cells/μL | | |
| | Placebo | Benra | | Placebo | Benra | |
| | (n = 64) | (n = 141) | P-value | (n = 133) | (n = 303) | P-value |
| Annual exacerbation rate | 0.88 ± 1.4 | 0.74 ± 1.4 | 0.12 | 1.10 ± 1.9 | 0.46 ± 1.2 | <0.0001 |
| ΔFEV₁ from baseline [L] | 0.02 ± 0.33 | 0.08 ± 0.31 | 0.39 | 0.05 ± 0.42 | 0.22 ± 0.40 | 0.0005 |
| ΔFVC from baseline [L] | 0.01 ± 0.51 | −0.02 ± 0.32 | 0.17 | 0.03 ± 0.42 | 0.08 ± 0.35 | 0.07 |
| | Cluster 5: Severe obstructed (n = 861) | | | | | |
| | Blood eosinophil counts | | | Blood eosinophil counts | | |
| | <300 cells/μL | | | ≥300 cells/μL | | |
| | Placebo | Benra | | Placebo | Benra | |
| | (n = 99) | (n = 172) | P-value | (n = 99) | (n = 394) | P-value |
| Annual exacerbation rate | 1.60 ± 2.1 | 0.98 ± 1.5 | 0.0048 | 1.80 ± 2.7 | 0.82 ± 1.4 | <0.0001 |
| ΔFEV₁ from baseline [L] | 0.12 ± 0.40 | 0.19 ± 0.38 | 0.06 | 0.31 ± 0.50 | 0.50 ± 0.55 | <0.0001 |
| ΔFVC from baseline [L] | 0.02 ± 0.36 | 0.08 ± 0.38 | 0.25 | 0.19 ± 0.48 | 0.35 ± 0.61 | 0.008 |

Data are mean ± SD.
P-values for all variables were derived with the Kruskal-Wallis test.
^{a}Combined data for benralizumab 30 mg every 4 weeks (Q4W) and 30 mg every 8 weeks (first three doses Q4W) treatment groups.

Cluster 2, an early-onset cluster, had an annual exacerbation rate reduction of −24% (p=0.08). Cluster 4, another early onset-cluster, had an annual exacerbation rate reduction of −34% (p=0.28). Cluster 3 and Cluster 5 (both late-onset clusters), had annual exacerbation rate reductions of −57% (p<0.0001) and −53% (p<0.0001), respectively.

Lung function improvements were greater with benralizumab vs. placebo in

Clusters 3, 4, and 5 while maintaining effect on postbronchodilator response (see FIGS. 4 and 5). Cluster 3 (+130 mL; p=0.0005) and Cluster 5 (+160 mL; p<0.0001) had the greatest improvements in FEV₁ with benralizumab vs. placebo from baseline. Cluster 5 FEV₁ increased 410 mL vs. baseline (p<0.0001).

### CONCLUSIONS

According to cluster analysis with the 11 baseline clinical variables identified from SARP clusters, patients with asthma from SIROCCO/CALIMA studies were grouped into Clusters 2, 3, 4, and 5. Most patients were assigned to Clusters 3 and 5, which are associated with late-onset, severe asthma. While Benralizumab treatment resulted in reductions in annual exacerbation rate across all clusters, reductions were greater for Cluster 3 and Cluster 5 (patients with late-onset disease) compared with Clusters 2 and 4 (early-onset disease) when assessed across baseline blood eosinophil counts. Treatment with benralizumab also improved prebronchodilator lung function, expressed as FEV₁ % predicted and FVC % predicted, across all clusters, with large improvements for Cluster 5 (patients with severe obstructed airway disease). Together with FVC improvements, improvements in FEV₁ (% predicted) while maintaining effect on post-BD response suggest changes in airway remodeling and/or mucus plugging leading to reduced airflow obstruction. In general, with stratification of clusters by baseline blood eosinophil counts, lung function improvements and exacerbation rate following benralizumab treatment were further improved for patients with ≥300 cells/μL vs. those with <300 cells/μL.

This study shows that an enhanced response to treatment with benralizumab, as evidenced by greater reductions in AER and improved lung function, was observed in late-onset asthma patients falling within SARP Clusters 3 and 5 compared to SARP Clusters 2 and 4.

Those skilled in the art will recognize, or be able to ascertain using no more than routine experimentation, many equivalents to the specific aspects of the disclosure described herein. Such equivalents are intended to be encompassed by the following claims.

Various publications are cited herein, the disclosures of which are incorporated by reference in their entireties.

Although the foregoing invention has been described in some detail by way of illustration and example for purposes of clarity of understanding, it will be obvious that certain changes and modifications can be practiced within the scope of the appended claims.

SEQUENCE LISTING
(Sequence 1 from US 20100291073)
Organism: Homo sapiens
SEQ ID NO: 1
DIQMTQSPSSLSASVGDRVTITCGTSEDIINYLNWYQQKPGKAPKLLIYHTSRLQSGVP
SRFSGSGSGTDFTLTISSLQPEDFATYYCQQGYTLPYTFGQGTKVEIK
(Sequence 2 from US 20100291073)
Organism: Homo sapiens
SEQ ID NO: 2
DIQMTQSPSSLSASVGDRVTITCGTSEDIINYLNWYQQKPGKAPKLLIYHTSRLQSGVP
RSFSGSGSGTDFTLTISSLQPEDFATYYCQQGYTLPYTFGQGTKVEIKRTVAAPSVFIF
PPSDEQLKSGTASVVCLLNNFYPREAKVQWKVDNALQSGNSQESVTEQDSKDSTYSLSS
TLTLSKADYEKHKVYACEVTHQGLSSPVTKSFNRGEC
(Sequence 3 from US 20100291073)
Organism: Homo sapiens
SEQ ID NO: 3
EVQLVQSGAEVKKPGASVKVSCKASGYTFTSYVIHWVRQRPGQGLAWMGYINPYNDG
TKYNERFKGKVTITSDRSTSTVYMELSSLRSEDTAVYLCGREGIRYYGLLGDYWGQG
TLVTVSS
(Sequence 4 from US 20100291073)
Organism: Homo sapiens
SEQ ID NO: 4
EVQLVQSGAEVKKPGASVKVSCKASGYTFTSYVIHWVRQRPGQGLAWMGYINPYNDGT
KYNERFKGKVTITSDRSTSTVYMELSSLRSEDTAVYLCGREGIRYYGLLGDYWGQGTLV
TVSSASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTCPPCPA
PELLGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYS
KLTVDKSRWQQGNVFSCSVMHEALHNHYTQKSLSLSPGK
(Sequence 5 from US 20100291073)
Organism: Homo sapiens
SEQ ID NO: 5
DLLPDEKJSLLPPVNFTIKVTGLAQVLLQWKPNPDQEQRNVNLEYQVKINAPKEDDYET
RITESKCVTILHKGFSASVRTILQNDHSLLASSWASAELHAPPGSPGTSIVNLTCTTNT
TEDNYSRLRSYQVSLHCTWLVGTDAPEDTQYFLYYRYGSWTEECQEYSKDTLGRNIACW
FPRTFILSKGRDWLAVLVNGSSKHSAIRPFDQLFALHAIDQINPPLNVTAEIEGTRLSI
QWEKPVSAFPIHCFDYEVKIHNTRNGYLQIEKLMTNAFISIIDDLSKYDVQVRAAVSSM
CREAGLWSEWSQPIYVGNDEHKPLREWFVIVIMATICFILLILSLICKICHLWIKLFPP
IPAPKSNIKDLFVTTNYEKAGSSETEIEVICYIEKPGVETLEDSVF
(Sequence 6 from US 20100291073)
Organism: Mus musculus
SEQ ID NO: 6
DLLNHKKFLLLPPVNFTIKATGLAQVLLHWDPNPDQEQRHVDLEYHVKINAPQEDEYDT
RKTESKCVTPLHEGFAASVRTILKSSHTTLASSWVSAELKAPPGSPGTSVTNLTCTTHT
VVSSHTHLRPYQVSLRCTWLVGKDAPEDTQYFLYYRFGVLTEKCQEYSRDALNRNTACW
FPRTFINSKGFEQLAVHINGSSKRAAIKPFDQLFSPLAIDQVNPPRNVTVEIESNSLYI
QWEKPLSAFPDHCFNYELKIYNTKNGHIQKEKLIANKFISKIDDVSTYSIQVRAAVSSP
CRMPGRWGEWSQPIYVGKERKSLVEWHLIVLPTAACFVLLIFSLICRVCHLWTRLFPPV
PAPKSNIKDLPVVTEYEKPSNETKIEVVHCVEEVGFEVMGNSTF
VH CDR1
SEQ ID NO: 7
SYVIH
VH CDR2
SEQ ID NO: 8
YINPYNDGTKYNERFKG
VH CDR3
SEQ ID NO: 9
EGIRYYGLLGDY
VL CDR1
SEQ ID NO: 10
GTSEDIINYLN
VL CDR2
SEQ ID NO: 11
HTSRLQS
VL CDR3
SEQ ID NO: 12
QQGYTLPYT

1. A method of treating a patient with late-onset asthma, comprising administering to said patient a therapeutically effective amount of benralizumab or an antigen-binding fragment thereof.

2. A method of treating a patient with asthma that falls within Severe Asthma Research Program (SARP) clinical cluster 3 or 5, comprising administering to said patient a therapeutically effective amount of benralizumab or an antigen-binding fragment thereof.

3. A method of reducing the annual exacerbation rate (AER) in a patient with late-onset asthma, comprising administering to said patient a therapeutically effective amount of benralizumab or an antigen-binding fragment thereof, wherein the administration reduces the patient's AER.

4. A method of reducing the AER in a patient with asthma that falls within SARP clinical cluster 3, comprising administering to said patient a therapeutically effective amount of benralizumab or an antigen-binding fragment thereof, wherein the administration reduces the patient's AER.

5. A method of reducing the AER in a patient with asthma that falls within SARP clinical cluster 5, comprising administering to said patient a therapeutically effective amount of benralizumab or an antigen-binding fragment thereof, wherein the administration reduces the patient's AER.

6. The method of claim 3, wherein the AER is reduced by at least 45% compared to a patient not administered the benralizumab or antigen-binding fragment thereof.

7. The method of claim 6, wherein the AER is reduced by at least 50% compared to a patient not administered the benralizumab or antigen-binding fragment thereof.

8. A method of improving lung function in a patient with late-onset asthma, comprising administering to said patient a therapeutically effective amount of benralizumab or an antigen-binding fragment thereof.

9. A method of improving lung function in a patient with asthma that falls within SARP clinical cluster 3, comprising administering to said patient a therapeutically effective amount of benralizumab or an antigen-binding fragment thereof.

10. A method of improving lung function in a patient with asthma that falls within SARP clinical cluster 5, comprising administering to said patient a therapeutically effective amount of benralizumab or an antigen-binding fragment thereof.

11. The method of claim 8, wherein the improved lung function is measured by an increase in the patient's percent predicted forced expiratory volume in 1 second (FEV₁) compared to the patient's FEV₁ prior to the administration.

12. The method of claim 11, wherein the FEV1 is pre-bronchodilator (BD) FEV₁.

13. The method of claim 12, wherein the pre-BD FEV₁ is increased by at least 6%.

14. The method of 12, wherein the pre-BD FEV₁ is increased by at least 14%.

15. The method of claim 11, wherein the FEV₁ is post-bronchodilator (BD) FEV₁.

16. The method of claim 15, wherein the post-BD FEV₁ is increased by at least 2%.

17. The method of claim 15, wherein the post-BD FEV₁ is increased by at least 10%.

18. The method of any claim 8, wherein the improved lung function is measured by an increase in the patient's percent predicted forced vital capacity (FVC) compared to the patient's FVC prior to the administration.

19. The method of claim 18, wherein the FVC is pre-bronchodilator (BD) FVC.

20. The method of claim 19, wherein the pre-BD FVC is increased by at least 6%.

21. The method of claim 19, wherein the pre-BD FVC is increased by at least 12%.

22. The method of claim 18, wherein the FVC is post-BD FVC.

23. The method of claim 22, wherein the post-BD FVC is increased by at least 1%.

24. The method of claim 22, wherein the post-BD FVC is increased by at least 7%.

25. The method of claim 2, wherein the SARP clinical cluster has been determined for the patient's asthma prior to the administration.

26. The method of claim 2, further comprising determining the SARP clinical cluster of the patient's asthma prior to the administration.

27. The method of claim 2, wherein the determination of the SARP clinical cluster is based on the age of asthma onset, pre-BD FEV₁, and post-BD FEV₁.

28. The method of claim 27, wherein the patient's age of asthma onset is 47±9.4 years, baseline FEV₁ is 66±7.7%, and maximal post-BD FEV₁ is 78±12%.

29. The method of claim 27, wherein the patient's age of asthma onset is 33±17.0 years, baseline FEV₁ is 43±9.4%, and maximal post-BD FEV₁ is 56±15%.

30. The method of claim 2, wherein the determination of the SARP clinical cluster is based on FEV₁, FVC, FEV₁/FVC, maximal post-BD FEV₁, maximal post-BD FVC, percentage change in post-BD FEV₁, age at asthma onset, asthma duration, patient gender, frequency of β2-agonist use, and inhaled corticosteroid (ICS) dosage.

31. The method of claim 2, wherein:
the age of asthma onset is 47±9.4 years;
the patient's baseline FEV₁ is 66±7.7%;
the patient's baseline FVC is 82±11%;
the patient's baseline FEV₁/FVC is 0.65±0.10;
the patient's maximal post-BD FEV₁ is 78±12%;
the patient's maximal post-BD FVC % is 91±14%;
the patient's change in post-BD FEV₁ is 0.22+/−0.40; and/or
the asthma has had a duration of 10±7 years.

32. The method of claim 2, wherein:
the age of asthma onset is 33±17.0;
the patient's baseline FEV₁ is 43±9.4;
the patient's baseline FVC is 65±12%
the patient's baseline FEV₁/FVC is 0.54±0.12;
the patient's maximal post-BD FEV₁ is 56±15%;
the patient's maximal post-BD FVC % is 77±15%;
the patient's change in post-BD FEV₁ is 0.50+/−0.55; and/or
the asthma has had a duration of 21±15 years.

33. The method of claim 1, wherein the patient has a baseline blood eosinophil count of >300 cells/μL prior to the administration.

34. The method of claim 133, wherein AER, FEV₁, and FVC values are improved in the patient compared to patients not administered the benralizumab or an antigen-binding fragment thereof.

35. The method of claim 1, wherein said patient has severe asthma.

36. The method of claim 33, wherein severe asthma is characterized by a requirement for treatment with high-dose ICSs and/or treatment with continuous or near continuous oral corticosteroids (OCs); and two or more of the following criteria: a requirement for additional daily treatment with a controller medication; asthma symptoms requiring short-acting β₂ agonist (SABA) use on a daily or near-daily basis; persistent airway obstruction; one or more urgent care visits for asthma per year; three or more oral steroid bursts per year; prompt deterioration with a ≤25% reduction in oral or inhaled corticosteroid dose; and/or near-fatal asthma event in the past.

37. The method of claim 1, wherein the benralizumab or antigen-binding fragment thereof is administered at about 30 mg per dose.

38. The method of claim 1, comprising administering at least two doses of the benralizumab or an antigen-binding fragment thereof to the patient.

39. The method of claim 1, wherein the benralizumab or antigen-binding fragment thereof is administered once every four weeks or once every eight weeks.

40. The method of claim 1, wherein the benralizumab or antigen-binding fragment thereof is administered once every four weeks.

41. The method of claim 1, wherein the benralizumab or antigen-binding fragment thereof is administered once every four weeks for twelve weeks and then once every eight weeks.

42. The method of claim 1, wherein the benralizumab or antigen-binding fragment thereof is administered parenterally.

43. The method of claim 42, wherein the benralizumab or antigen-binding fragment thereof is administered subcutaneously.

44. The method of claim 1, wherein the benralizumab or antigen-binding fragment thereof is administered in addition to corticosteroid therapy and/or short- or long-acting B₂-agonist therapy.

45. The method of claim 1, wherein the patient has an asthma control questionnaire score of at least 1.5 prior to the administration of benralizumab or antigen-binding fragment thereof

46. A method of predicting an asthma patient's therapeutic response to benralizumab or an antigen-binding fragment thereof, the method comprising, determining, prior to administration of the benralizumab or antigen-binding fragment thereof, the SARP clinical cluster of the asthma.

47. The method of claim 46, comprising predicting an enhanced response to the benralizumab or antigen-binding fragment thereof if the SARP clinical cluster is determined to be cluster 3 or cluster 5.

48. The method of claim 46 or 47, further comprising administering the benralizumab or antigen-binding fragment thereof to the patient if the SARP clinical cluster of the patient's asthma is determined to be cluster 3 or cluster 5.
